(19) United States
(12) Patent Application Publication
(10) Pub. No.: US 2019/0159721 A1
(43) Pub. Date: May 30, 2019

(54) ASSISTED CAPACITY DEVICE CALCULATOR FOR MYOCARDIAL INFARCTION (AC/DC for MI)

(71) Applicant: Katrina Goff CANDY, Moama (AU)

(72) Inventor: Katrina Goff CANDY, Moama (AU)

(21) Appl. No.: 16/252,624

(22) Filed: Jan. 19, 2019

Related U.S. Application Data
(63) Continuation-in-part of application No. 15/399,103, filed on Jan. 5, 2017.

(30) Foreign Application Priority Data
Aug. 29, 2016 (AU) 2016903415

(51) Int. Cl. A61B 5/00; A61B 5/0402; A61B 5/145; A61B 5/04

(52) U.S. Cl. CPC A61B 5/486; A61B 5/0022; A61B 5/0402; A61B 5/04012; A61B 5/742; A61B 5/7475; A61B 5/681; A61B 5/14546

(57) ABSTRACT

The assisted capacity device calculator for myocardial infarction system is intended to assist a user during, or recently having had, a myocardial infarction and is intended to aid to help all people, especially those who have are susceptible to having myocardial infarctions, i.e., heart attacks. The apparatuses of the system are predominately mobile but can also be stationary and can be programmed by receiving and selecting pre-set commands to operate and assist a user but is particularly adapted for use when a user is having symptoms of a myocardial infarction. Particularly, the system is capable of prompting the user to respond to myocardial infarction symptoms, by answering various symptom questions, and if necessary, activate electrocardiographic (ECG) components, to test for electro cardio activity and/or a blood analyzer sensors to test for cardiac markers. The apparatuses of the also are provided with various features including an illuminated display panel, a CPS tracking capability, alarms among other things.

### CROSS-REFERENCE TO RELATED APPLICATIONS

This application is a continuation-in-part of U.S. patent Non-Provisional application Ser. No. 15/399,103 filed Jan. 5, 2017 which is a continuation of Australian provisional application 2016903415 of Katrina Goff Candy filed Aug. 29, 2016.

### FEDERALLY SPONSORED RESEARCH AND DEVELOPMENT

None.

### APPENDICES

None.

### BACKGROUND OF THE INVENTION

These is a need for an apparatus which has the capability to assist individuals in monitoring their health by determining how much food, exercise and rest they are getting on a daily basis. All of these factors affect an individual's weight and a healthy diet and exercise routine will both ensure proper weight gain or loss by preventing overeating and getting proper exercise. And as everyone is unique in terms of age, body type, body mass index, i.e., the ratio of fat to overall weight, metabolic rates and activity levels, homeostatic balances, etc., the number of calories a person ingests in the way of food and expends in calories, in the form of normal body function and exercise on a daily basis will vary. Additionally, appropriate water and hydration intake can vary by individual and can also be a critical element in attaining and maintaining a healthy body. Therefore, eating the proper amount and types of food versus the kinds and amounts of exercise one incurs daily will have a critical impact on one's weight and overall health.

In addition, modern medical science indicates that one of the most dangerous diseases for the human race is heart disease, notably heart diseases attributed to atherosclerosis. Atherosclerosis is attributed to cholesterol accumulation which, if untreated, results in plague buildup in coronary arteries and many cases lead to “sudden” heart attacks when the plaque ruptures causing thrombosis blood flow blockage. These blockages lead to oxygen starvation in cardiomyocytes; if these conditions persist, even for a relevantly short time, permanent heart muscle damage can result. Additionally, these “sudden” heart attacks often occur with little or no warning to a person and can lead to major heart damage and/or death.

### SUMMARY OF THE INVENTION

For the purposes of the following disclosure, the term “user, User, user(s), or User(s)” denotes a person or persons who is using the invention system, apparatuses, or components as described in the instant invention; occasionally, in the disclosure, the term “patient/user” maybe is used to denote a “patient” or a “user”. Similarly, the term “carer” refers to any number of health professionals or other individuals involved with providing any time of care, mental, physical, or otherwise for a “user” and/or “patient”.

The assisted capacity device calculator for myocardial infarction system, hereinafter the “AC/DC for MI system” comprises a number of apparatuses and components which co-operate to assist a user in monitoring basic human body parameters capable of determining whether the User is, or has recently had, a cardiac infarction, i.e., a heart attack. The AC/DC for MI system is provided with AC/DC for MI apparatuses capable of monitoring the User's body temperature, heart rate, respiratory rate, along with blood pressure and fluid values such as red/white body cell counts, glucose levels, blood protein levels, electro cardio activity, etc. The AC/DC for MI system is further provided with a number of components which co-operate with the AC/DC for MI apparatuses to affect the operation of the system. The AC/DC for MI system, apparatuses and components are further capable of updating the human body parameters using software computer applications imbedded in the AC/DC for MI apparatuses by micro-chip technology. The AC/DC for MI apparatuses and components allow the AC/DC for MI system to diagnose the User to determine if he/she is having a heart attack. Additionally, the AC/DC for MI system is capable of prompting and/or alerting a user as to a need for action by various means such a screen displays, vibrations, sound alerts, etc. The need for action by a user may involve responding to a series of questions or prompting a user utilize electrocardiographic sensors and/or blood sensors.

The AC/DC for MI system also has a copy of the User's medical records and genetic genome to allow for instant access to the user total medical and genetic history especially in emergency medical situations. Most importantly the AC/DC for MI system is provided with at least one reference electrocardiogram, (ECG) for the User which can be immediately analyzed with an ECG monitor for determining an assessment of a potential heart attack episode.

Particularly, the AC/DC for MI system, during a typical use, asks the User various questions symptomatic of heart attacks and incorporates any responses in its database for heart attack analysis. For example, the User is asked if she/he have incurred chest pains such a heavy load on their chest, some liken it to a brick being place on their chest; additionally, the User is asked whether these pain(s) radiate to the arms, particularly the left arm and/or to their jaw area. The User would be further asked if they feel nauseated, have vomited, feel dizzy, have lost consciousness and/are short of breath. Finally, the User would be asked how long these symptoms have persisted, as time frames are critical in diagnosing heart attacks; for example, an attack lasting longer than 20 minutes can cause permanent heart muscle loss.

Additionally, the AC/DC for MI system incorporates an electronic pedometer capable of measuring the user activity level such as distance traveled, intensity level, height or altitude, etc. and measures total calories expended at any given time.

The AC/DC for MI system is also provided with an electrocardiographic sensors also known as ECG sensors, which records a User's heart electrical activity, i.e., electro cardio activity, for a period of time, i.e., an ECG. The ECG sensors comprise electrodes placed on a patient's body which can detect the tiny electrical changes on a person skin. By comparing an ECG taken during a heart attack episode to a reference ECG in the User's medical records, stored in the AC/DC for MI system, the resulting comparison between the ECGs can be used to assist in determining whether the person is, in fact, having a heart attack.

Finally, the AC/DC for MI system is provided with means for analyzing a user's blood sample to determine any biochemical signs, or evidence, of a heart attacks commonly known as “cardiac markers”. When a heart attack occurs, cardiomyocytes begin to break down releasing foreign proteins in a user's bloodstream. For example, a typical protein released during a heart attack is troponin; similarly, creatine kinase may be released along with myoglobin; all of these biochemicals can be readily identified as symptomatic of cardiac attacks. As the AC/DC for MI system completes its analysis of the above three studies, any two of which are highly indicative of an ongoing heart attack, an alert can be triggered for the User to seek immediate medical assistance.

Homeostats, in the body, are types of negative biological feedback mechanisms in the body used to regulate various body systems. For example, to regulate and maintain the body at 98.6 degrees Fahrenheit, the normal body temperature, a negative feedback loop in the form of your skin nerve cells sends a message to your brain if you the temperature goes above the normal body temperature. The brain subsequently sends a message to sweat glands to open and release water thereby bringing the body temperature down to the normal temperature. The AC/DC for MI system is further constructed to be programmed with the user's metabolic analysis and various homeostatic analyses including body core temperature homeostat along with blood related measures such as blood glucose and oxygen content homeostat, blood partial pressure of oxygen/carbon dioxide homeostat, blood oxygen content homeostat, blood arterial blood pressure homeostats as well as plasma ionized calcium homeostat. Additionally, it is also critical to assess the user's extracellular sodium and potassium concentration along with volume of body water and extracellular fluid pH homeostats.

Among other things, the AC/DC for MI system is constructed to record, monitor and show a user their daily food intake and make suggestions to the user regarding healthy food choices. For example, when one is planning a meal, choosing a snack reading food labels is critical in determining proper food choices. Additionally, the amount and type of daily ingested nutrients, be it macro nutrients, i.e. water, protein, carbohydrates, fats, fiber, or micro nutrients, i.e., vitamins and minerals, must be carefully analyzed and recorded by the user. And as nutrients generally provide material for assisting and maintaining cellular growth, providing body energy, in the form of calories, and other for body functions.

Energy for the body is generated primarily by proteins and carbohydrates which during the metabolic process produces sugars which are the main source for energy in the human body. Other sugars, usually in the form of fructose, naturally occurring sugar in fruit for example, are also a major source of energy for human. For many individuals, foods containing added sugar, many times in the form of sucrose, versus naturally occurring sugar are particularly problematic for attaining and maintaining proper body weight. Reducing and controlling the amount of added sugar is a major approach of preventing diseases such as diabetes.

In addition to nutrition, another critical factor in attaining and maintaining body weight and health is a regular exercise routine. This may involve simple household chores such a cooking, cleaning, etc. but may also include dedicated routines such as walking, running, cycling, gym or health center regiments. Finally, a major component to well-being includes sufficient among of sleep which for children is particularly essential to ensure growth and health while in adults the proper among of sleep minimizes various health problems. The AC/DC for MI system pedometer is capable of measuring these activities and the AC/DC for MI system software is constructed to calculate and monitor the user's daily calorie expenditure.

Other factors that may affect an individual's weight and overall health is the amount of muscle mass produced by weight training, medication, disease and for women natural conditions such menopause or pregnancy. Additionally, the AC/DC for MI system is constructed to monitor other physical body attributes such a walking gait and posture and to alert the user in event of changes in those attributes.

As a typical example for use the AC/DC for MI system, the User would be assessed or have an assessment conducted to determine age, BMI, weight, height, etc. along with a biochemical/physiological evaluation as part of an overall health profile including vital signs, i.e., heart rate and pressure, respiratory rates, ECG, etc. A critical factor to be assessed is a person's resting metabolic rate. Additionally, there would be a biochemical/physiological evaluation, among other things, which would measure a person's white blood count, hemoglobin, hematocrit and platelet count along with sodium, potassium and fasting glucose, triglyceride, cholesterol, and liver enzymes levels, etc. These evaluations are measured against normal ranges for similar individuals of age, gender, etc. Subsequently, a regiment would be developed by the AC/DC for MI system for the user for food intake and exercise daily and the as the user progresses during the day the of day the AC/DC for MI system monitors the user. Any adjustment of food and or other activities for the user would then be calculated and provided to the user to make appropriate adjustments. As human beings are endotherms in the animal kingdom, that is their bodies have metabolic rates which produce sufficient energy to heat up and maintain their body temperature at a relative constant temperature, usually 98.6 degrees Fahrenheit. For male humans, on average, their bodies require between 1600 to 1800 kilocalories of energy per day to function normally while female humans require between 1300 to 1500 kilocalories of energy per day. These are daily requirements for human at rest; obviously the more active an individual, the greater amount of energy is required.

The AC/DC for MI system is further constructed to either be mobile or stationary and may come in various applications, e. g, such as a cord necklace, or lanyard fitted AC/DC for MI apparatus, to be worn by the user. The AC/DC for MI system is also constructed to be fitted to a wrist bracelet or watch band and is further provided with a camera intake/scanner feature for scanning food or other labels to determine caloric and other nutrition information.

### BRIEF DESCRIPTION OF THE DRAWINGS

FIG. 1 discloses a diagram of a human heart of a user for use with the AC/DC for MI system.

FIG. 2 is a concept flow chart showing how the AC/DC for MI system functions to perform its operation.

FIG. 3 is a detailed figure of a first embodiment of the AC/DC for MI system disclosing its microchip and power.

FIG. 3A is a front view of the first embodiment of the AC/DC for MI system showing features of the first embodiment of the AC/DC for MI system.

FIG. 3B is a side view of the first embodiment of the AC/DC for MI system showing features of the first embodiment of the AC/DC for MI system.

FIG. 3C is a cut-away view of the first embodiment of the AC/DC for MI system of FIG. 3A.

FIG. 3D is a front view of the components of the AC/DC for MI apparatus of FIG. 3A. system showing an additional feature of the first embodiment of the AC/DC for MI system.

FIG. 3E is a side view of the components of the AC/DC for MI apparatus of FIG. 3D.

FIG. 4 is a detailed figure of the AC/DC for MI apparatus of FIG. 3 showing features of the first embodiment of the AC/DC for MI system.

FIG. 4A is a front view of the AC/DC for MI apparatus of FIG. 3.

FIG. 4B is a rear view of the AC/DC for MI apparatus of FIG. 3.

FIG. 4C is a front view of the AC/DC for MI apparatus of FIG. 3 showing an additional feature of the first embodiment of the AC/DC for MI system.

FIG. 4D is a front view of components of the AC/DC for MI apparatus of FIG. 3 showing an additional feature of the first embodiment of the AC/DC for MI system.

FIG. 5 discloses further features of the embodiment of the AC/DC for MI apparatus of FIGS. 3 and 4 for myocardial infarction and its operation with either a mobile electronic tablet and/or a mobile electronic smart phone.

FIG. 6 shows details of the operation of the AC/DC for MI system of FIG. 5.

FIG. 7 shows a second embodiment of the AC/DC for MI system.

FIG. 7A is a front view of the second embodiment of the AC/DC for MI system showing features of the second embodiment of the AC/DC for MI system.

FIG. 7B is a rear view of the second embodiment of the AC/DC for MI system showing features of the second embodiment of the AC/DC for MI system.

FIG. 7C is a cut away view of the second embodiment of the AC/DC for MI system showing features of the second embodiment of the AC/DC for MI system of FIG. 7A.

FIG. 8 shows further details of the AC/DC for MI apparatus of FIG. 7.

FIG. 9 shows further details of the AC/DC for MI apparatus of FIG. 7.

FIG. 10 shows the AC/DC for MI apparatus of FIG. 7 in use by a user.

FIG. 11 is a step by step flow chart of an example of the operation of the AC/CD for MI system.

FIG. 12 shows the set of questions to a patient for a heart attack prompted by the AC/DC for MI system.

FIG. 13 show ECG sensors and locations used to test for a heart attack with the AC/DC for MI system.

FIG. 13A show ECG sensor locations used to test for a heart attack with the AC/DC for MI system.

FIG. 13B show a front view of an ECG sensor used to test for a heart attack with the AC/DC for MI system.

FIG. 13C show a rear view of an ECG sensor used to test for a heart attack with the AC/DC for MI system.

FIG. 14 shows how the ECG sensors and their operation with either a mobile electronic tablet and/or a mobile electronic smart phone of the AC/DC for MI system.

FIG. 15 shows ECG displays used to test for a heart attack with the AC/DC for MI system.

FIG. 15A shows a user ECG base reference display used to test for a heart attack with the AC/DC for MI system.

FIG. 15B shows a user ECG test display used to test for a heart attack with the AC/DC for MI system.

FIG. 16 shows a side view of the blood sensor used to test for a heart attack with the AC/DC for MI system.

FIG. 17 shows a top view of the blood sensor used to test for a heart attack with the AC/DC for MI system.

FIG. 18 shows a top view of a second embodiment of the blood sensor used to test for a heart attack with the AC/DC for MI system.

FIG. 19 shows the various blood biochemicals tested for a heart attack using the blood sensor used to test for a heart attack with the AC/DC for MI system.

### DETAILED DESCRIPTION OF THE INVENTION

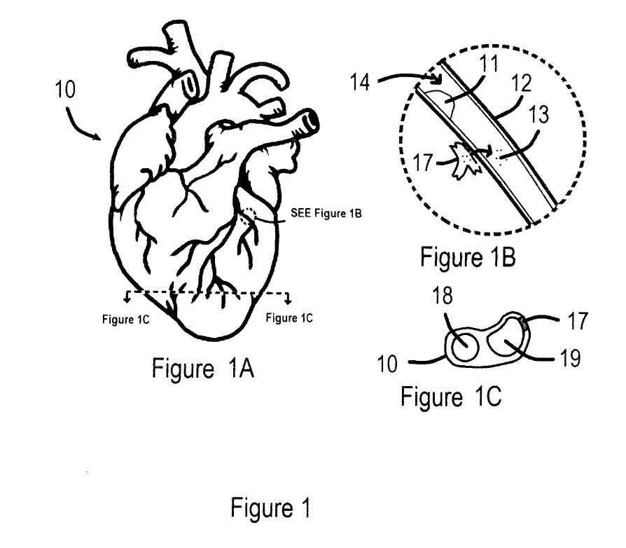

FIGS. 1, 1A, 1B and 1C show the pathology of a typical heart attack in a human being. Particularly, in FIG. 1A reveals a typical human heart 10 with FIG. 1B displaying a close up view of an artery 12 of heart 10. Particularly, in FIG. 1B the artery 12 may have cholesterol deposits 11 which may cause restrict blood flow 14 and result in areas of the arterial wall, of the heart, to rupture at myocardials 17 dispersing cardiomyocytes 13 into the blood flow. Cardiomyocytes 13 contain various cardiac markers, troponin, myoglobin, etc., or cardiac marker evidence, which can be analyzed to determine if a cardiac infarction is or has recently occurred. FIG. 1C is a cross-sectional view of heart 10 showing heart chambers 18 and 19 and particularly, weakened myocardial area 17.

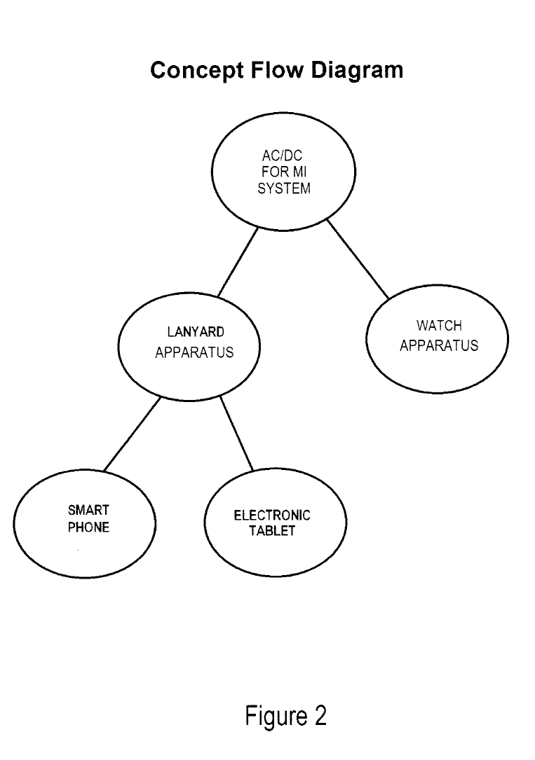

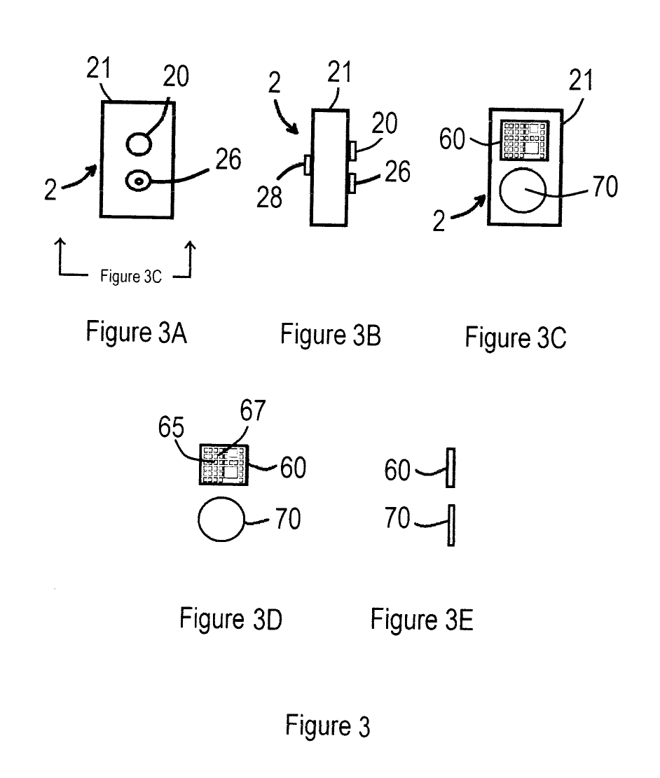

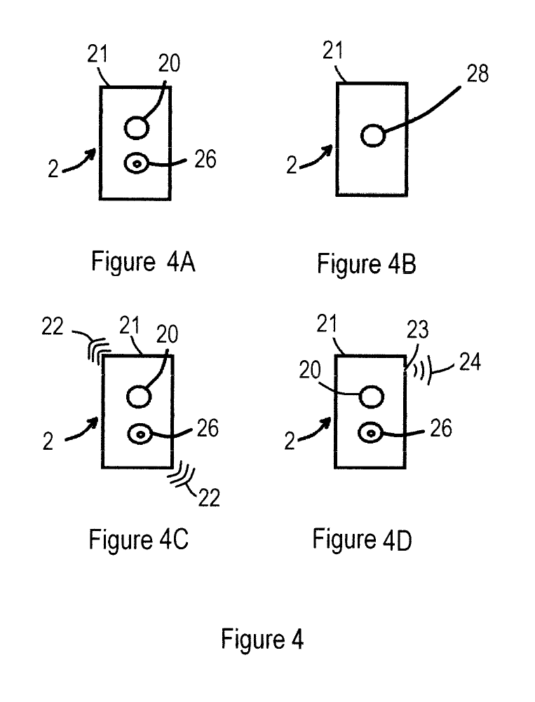

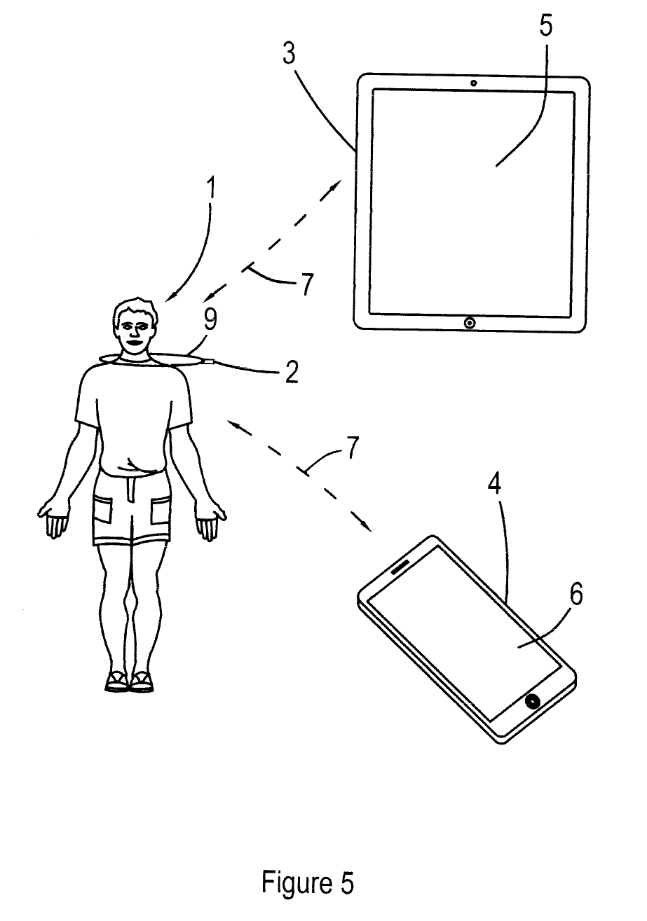

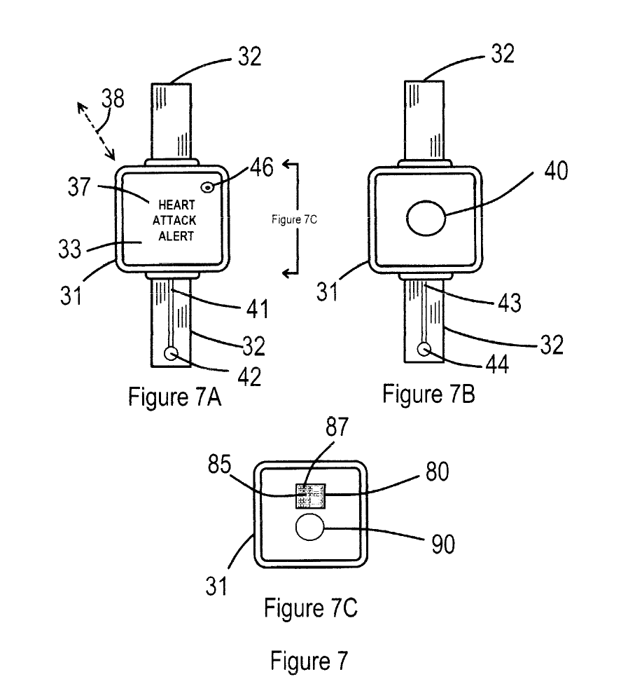

Now referring to FIG. 2, a concept flow diagram of the system according to the preferred embodiments of the invention is shown. Particularly in FIG. 2, the overall operation of the preferred embodiments of the invention are disclosed wherein the AC/DC for MI system, denoted by the encircled term apparatus may comprise a wrist watch, or lanyard attachment. As FIG. 2 furthers discloses, the AC/DC for MI system, whether it is be the AC/DC for MI apparatus of the of embodiment of FIG. 3-6, or 7-10, is provided with a AC/DC for MI sensor, as further discussed in reference to FIGS. 4 and 7, respectively, capable of creating and transmitting electronic signals, via a wireless signal transmitter imbedded in the AC/DC for MI sensor of the respective the AC/DC for MI apparatus. In the first embodiment, the wireless signals are transmittable to a mobile electronic appliance, from the AC/DC for MI apparatus 2, as discussed in FIGS. 3 and 5, such as a smart phone 3 or an electronic tablet 4. The mobile electronic appliances 3 and 4, are further capable of sending, receiving, and processing the wireless signals and/or data and are compatible with the Android mobile operating system, the iPhone cellular phone mobile operating system, i.e., iOS, and other commercially available mobile device operating systems. Additionally, the sensors of the respective AC/DC for MI apparatuses, as further discussed with reference to FIGS. 4 and 7, respectively incorporate radiofrequency microchip technology, in the form of a microcomputer transmitter processor capable of sending, receiving, processing, and transmitting data and/or signals to and from navigational positioning systems, such as the Global Positioning System for locating the sensors, and therefore the respective AC/DC for MI apparatus; in the case of the first embodiment of FIGS. 3 to 5, these signals and/or data, reference number 7 of FIG. 5, can transmit signals and/or data to the mobile electronic appliances 3 and/or 4. As is further shown in FIG. 5, the mobile electronic appliance 3 or 5, respectively, is provided with or is accessible to computer processing application software, i.e., otherwise known as an app, 5 of electronic device 3 and 6 of electronic 4, which app is capable of processing the any signal and/or data receiving by the respective sensor. The second embodiment of AC/DC for MI system shown and discussed reference to FIGS. 7 to 10, is also provided with sensor capable of transmitting and receiving signals and data for assessing, among other things, the physical condition and/or location of a user.

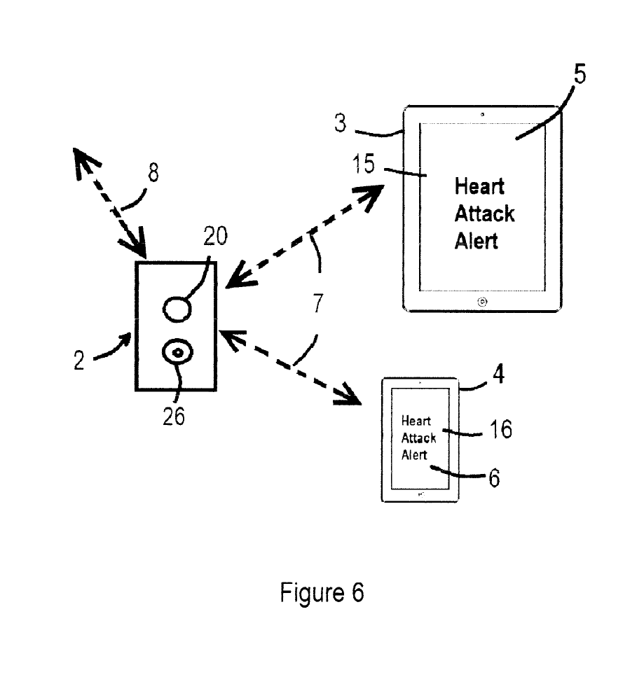

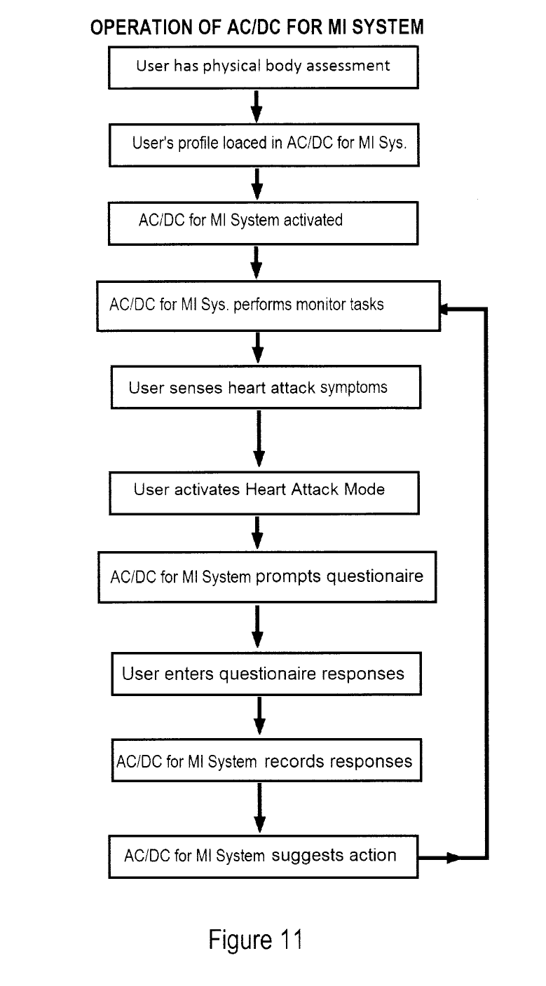

FIG. 3 discloses further details and operation of the first embodiment of AC/DC for MI apparatus 2. Particularly, FIG. 3A shows that AC/DC for MI apparatus 2 has a body having a body 21 of a rectangular cuboid structure sized to be held in the hand of a user. AC/DC for MI apparatus 2 is provided with an activation button 20 and a camera scanner 26; the button 20 which can be depressed by a User to activate apparatus 2. Additionally, apparatus 2 is capable of creating transmitting and receiving electronic signals 7, as shown in FIG. 5, via a wireless signal transmitter imbedded in apparatus 2 in the form of a microcomputer sensor transceiver processor 60, further disclosed and discussed in reference to FIG. 4C to 4E. Upon activation, microcomputer sensor transceiver processor 60 sends a wireless signal 7, (shown in FIGS. 5-6) by various systems, such as a technology that allows electronic devices to network, i.e., WI-FI, or a wireless technology standard for exchanging data over short distances commonly known as Bluetooth, or ZigBee, etc. Additionally, wireless signal 7 can be transmitted to an emergency alert system such medical hospital or clinic alert staff desk, nursing station, emergency office or other medical facility. The wireless signal 7 is also transmittable to electronic tablet 3 or a smart phone 4, also shown in FIG. 5. The electronic tablet 3 or smart phone 4 is also capable of sending, receiving, and processing the wireless signals 7 and/or data and also is compatible with the Android mobile operating system, the iPhone cellular phone mobile operating system, i.e., iOS, and other commercially available mobile device operating systems. Additionally, the microcomputer sensor transceiver processor 60 incorporates radiofrequency microchip technology capable of sending, receiving, and processing data and/or signals 8 to and from navigational positioning systems, such as the Global Positioning System, i.e., GPS, or the Global Navigational Satellite System, i.e., the GLONASS for geographically locating the microcomputer sensor transceiver processor 60. Microcomputer sensor transceiver processor 60 is further capable of being coded with information, in a software application, otherwise call an app 65, as shown in FIG. 3D, regarding the identity of the user such as his or her birth date, home location, medical records, and other the medical information of the user. The medical information may include, as discussed further in regards to FIG. 11, a physical body assessment. The AC/DC for MI apparatus 2 further incorporates a pedometer 67, in microcomputer sensor transceiver processor 60, as further shown in reference to FIG. 3D, in the form of a MEMS, i.e., a microelectromechanical system in the form of an inertial sensor capable of measuring 1 to 3 axis acceleration data, which data is processed by the app 65; the data from app 65 is capable of calculating expended User calorie levels based on the pedometer sensor data. App 65 is further programmed to receive input from the User and from data from the pedometer and sensor probe 28, discussed further in reference to FIG. 3B, and co-ordinates with app 5 of electronic tablet 3 and/or app 6 of electronic smartphone 6, to make various calculations regarding the user's medical and physical condition particularly caloric intake and expenditure. The AC/DC for MI apparatus 2 is also provided with a camera scanner 26 allowing the apparatus to either take and record pictures and photos or to scan bar codes particularly bar codes on food packages for inputting calorie and nutrition information into AC/DC for MI system. Finally, the AC/DC for MI apparatus 2 is programmed to be operated by either an app 5 of electronic tablet 3 or app 6, of electronic smartphone 6 to display or readout data of various functions of the AC/DC for MI apparatus, particularly to the measure heart rate, respiratory rate or temperature of a user, etc. Camera scanner 26 of AC/DC for MI apparatus 2, shown in FIGS. 3A-3B, allows the User to scan food labels to input calorie and nutrition information into the apparatus. The AC/DC for MI apparatus 2 as is discussed in reference to FIGS. 3-4 is shown as being powered by a battery, although the apparatus is can be configured to be with solar energy.

FIG. 4 reveals additional features of AC/DC for MI apparatus 2. Particularly FIG. 4A shows the overall construction of AC/DC for MI system apparatus 2 while FIG. 4B is a rear view of FIG. 4A revealing sensor probe 28 capable of, among other things, sensing a user's temperature, heart or respiratory rate and can be used to test for myocardiates. FIG. 4C reveals the vibration feature 22 of AC/DC for MI system allowing the apparatus to act in an alarm or awakening mode while FIG. 4D reveals a sound transmitter and receiver 23 provided with AC/DC apparatus 2 capable of producing alarm or awakening sound 24. Sound transmitter and receiver 23 is also capable of receiving voice commands or input for operating the AC/DC apparatus 2. As previously discussed, microcomputer sensor transmitter processor 60 is capable, via app 65, of being coded with identity, medical and physical body assessment information regarding the user, as discussed above, and is capable of creating and receiving signals and/or data which can be transmitted to a satellite navigational technology system as discussed in reference to FIG. 2, supra. Microcomputer sensor transceiver processor 60 also can transmit and receive signals and/or data to the mobile electronic appliance, electronic tablet 3 or smart phone 5 and is programmed with app 65 for analyzing and processing medical and physical body input data such a heart rate, body temperature or respiratory rates.

FIG. 5 shows further details of the first embodiment of the AC/DC for MI system particularly AC/DC for MI apparatus 2 being utilized by a user 1. In FIG. 5, the AC/DC for MI apparatus 2 is shown being attached to User 1 via lanyard or necklace 9 and the AC/DC for MI apparatus 2 is capable of electronically communicating, by wireless signals 7, as is disclosed and discussed below, with an electronic appliance in the form of an electronic tablet 3 having a visual screen 5 and/or smart phone device 4 having visual screen 6.

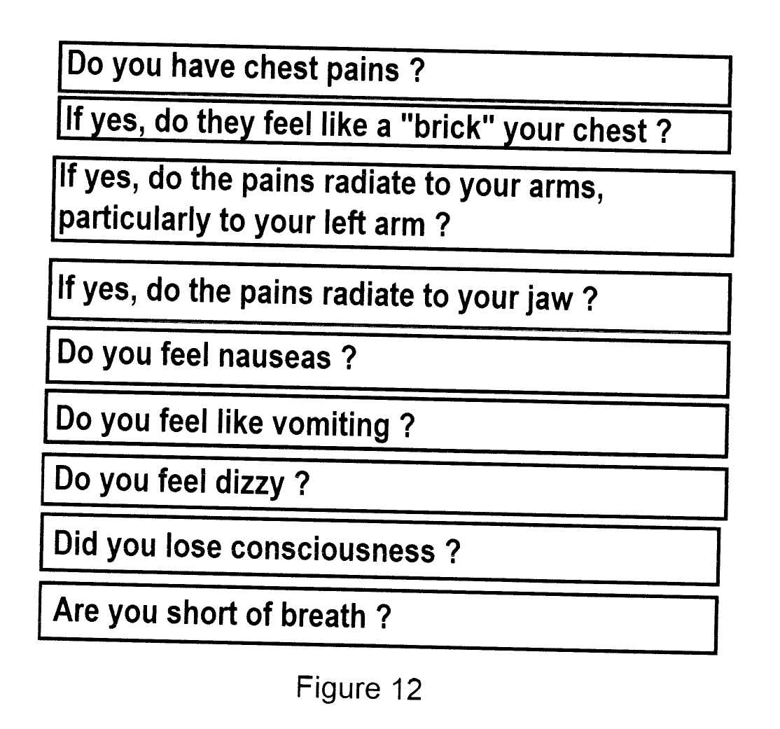

As is further shown in FIG. 6, a mobile electronic appliance, either electronic tablet 3 or smart phone 4, is provided with an app, not shown, which is capable of processing any signal and/or data received by a sensor device from the AC/DC for MI apparatus 2 and also capable of producing a readout or display on a visual screen display. For the electronic tablet 3, this readout or display is shown as reference number 15 on visual screen 5 while for the smart phone 4, the readout or display is shown as reference number 16 on visual screen 6. In the examples of FIG. 6, the readout or display is “Heart Attack Alert” and refers to have the AC/DC for MI apparatus 2 performing particular physical body attribute tasks, here to assess whether the User is having a myocardial infarction; the details of the attribute tasks are discussed in reference to FIGS. 12 to 19. Additionally, electronic tablet 3 and smart phone 4 are touch activated by the user to perform the various tasks desired by the User.

FIG. 7 discloses a second embodiment of the AC/DC for MI system. Particularly, FIG. 7A discloses an AC/DC for MI apparatus 31 housed in the form of watch type configuration, having watchband 32, wearable on a hand of a user. Additionally, as FIG. 7A discloses, AC/DC for MI apparatus 31 has a visual screen 33 and visual screen display readout 37, produced via an app 85, as shown and discussed in reference to FIG. 7C, imbedded in the AC/DC for MI apparatus 31 similar to that of AC/DC for MI apparatus 2 app 65. The visual screen display readout 37 is capable of displaying or showing a readout to a user here, as an example “Heart Attack Alert”. AC/DC for MI apparatus 31 is further configured to be touch activated on its screen by the user and can received input from a user. AC/DC for MI apparatus 31 can also be configured with traditional push buttons, not shown. Additionally, AC/DC for MI apparatus 31 has an illumination display feature allowing the for the AC/DC for MI apparatus to be seen at night or in low-light environments. Also, seen in FIG. 7A, is camera scanner 46 allowing scanning of food and dietary information, for input calorie and nutrition information similar to camera scanner 26 of AC/DC for MI apparatus 2. Further shown in FIG. 7A, is a sensor probe 42, similar to sensor probe 28 of AC/DC for MI apparatus 2, which sensor probe 42 is also capable of, inter alia, sensing a user's body temperature, heart pulse rate or respiratory rate; sensor probe 42 is connected to AC/DC for MI apparatus 31 by electrical connector 41. FIG. 7B is a rear view of the AC/DC for MI apparatus 31 and discloses sensor probe 40, sensor probe 44 which is connected to AC/DC for MI apparatus 31 by electrical connector 43. Sensor probes 40 and 42 have the same sensing capability as sensor probe 42. All of the data from sensor probe, 40, 42, and 44 are capable of being inputted into an app 85 located in microcomputer sensor transceiver processor 80, shown and further discussed in reference to FIG. 7C. Additionally, AC/DC for MI apparatus 31 and microcomputer sensor transceiver processor 80 also incorporates a MEMS, i.e., a microelectromechanical system in the form of inertial sensor capable of measuring 1 to 3 axis acceleration, i.e., a pedometer 87, shown and discussed in reference to FIG. 7C. App 85 is functionally similar to app 65 of AC/DC for MI apparatus 2 and also is programmed to receive input from a user at any time, via visual screen 33, from data from the pedometer 87 and sensor probes 40, 42, or 44, to make various calculations regarding a user's medical and physical condition particularly caloric intake and expenditure. Additionally, sensor probe 42 is located and positioned on band 32 such that can be held against the chest area of a user to measure the user's respiratory rate while sensor probe 44 is constructed and located on the AC/DC for MI apparatus 31 to sense the heart rate on the wrist of a user. Sensor probe 44 is further capable of sensing a user's body temperature. FIG. 7C, a cross-section view of AC/DC for MI apparatus 31 of FIG. 7A, reveals that AC/DC for MI apparatus 31 is internally provided with electronic components 80 and 90 similar to microcomputer sensor transceiver processor 60 and power source 70 of AC/DC for MI apparatus 2. As such, AC/DC for MI apparatus 31 is capable of electronically communicating with GPS and GLONASS satellite systems for allowing location of wearer of the AC/DC for MI apparatus 31 and, i.e., the user.

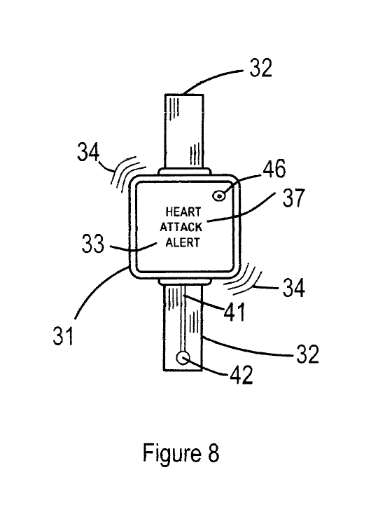

FIG. 8 discloses an additional feature of the AC/DC for MI apparatus 31 of FIG. 7 particularly, a vibration capability 34 allowing for AC/DC for MI apparatus 31 to remind and or alert the user to perform or to display a particular visual display readout, here, as an example, to “Heart Attack Alert”.

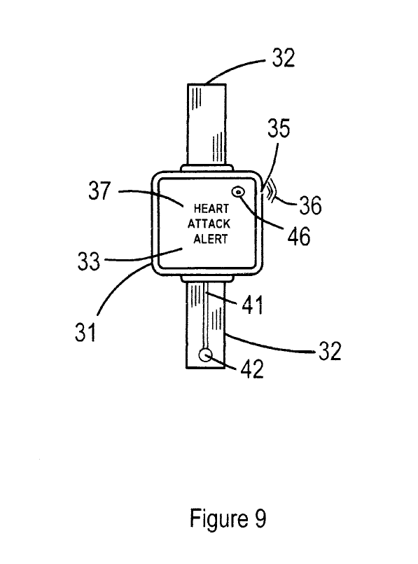

FIG. 9 discloses an additional feature of the AC/DC for MI apparatus 31 of FIG. 7, particularly, a sound transmitter and receiver 35 capable of emitting sound 36 for alerting the user to remind and or alert the user to perform or to display a particular visual display readout, here, as an example, to “Heart Attack Alert”. Additionally, transmitter and receiver 35 is capable of receiving audible input which can be used to program AC/DC for MI apparatus 31.

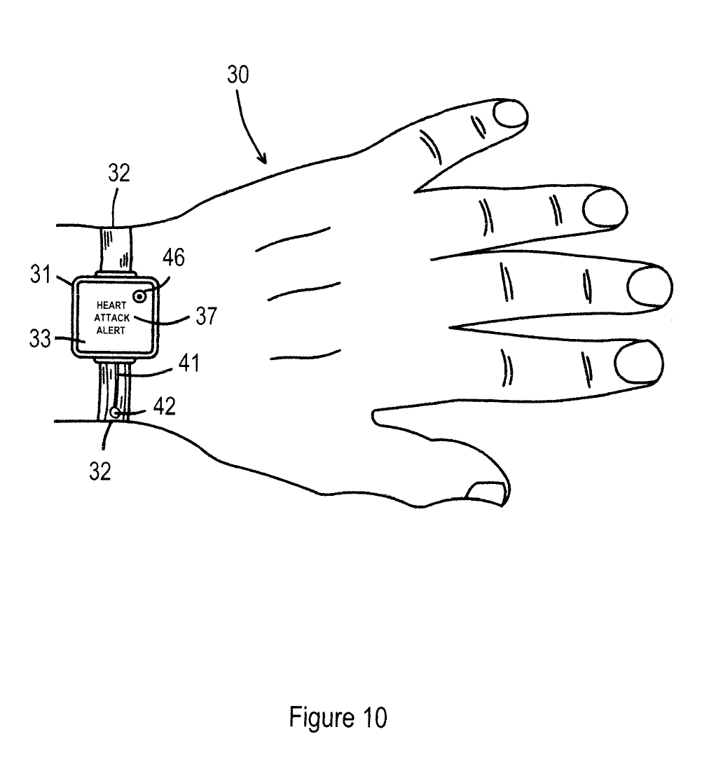

FIG. 10 shows the AC/DC for MI apparatus 31 of FIG. 7 being utilized by a user, i.e., being worn on the wrist of hand 30 of the User.

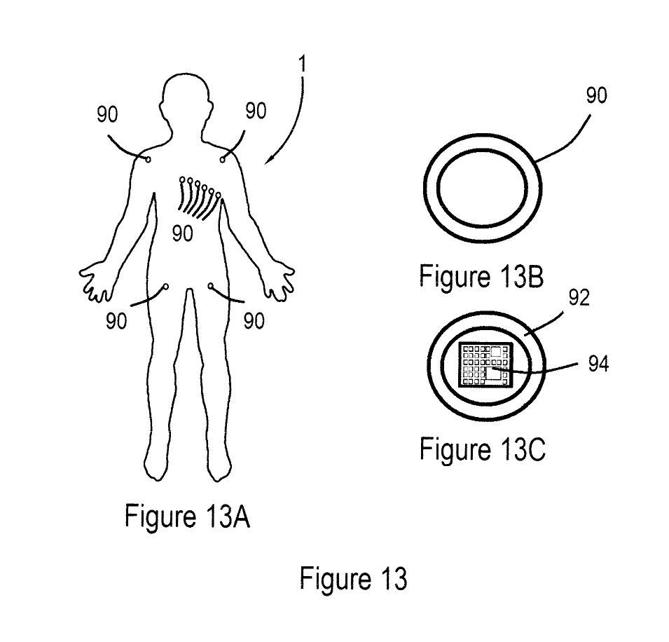

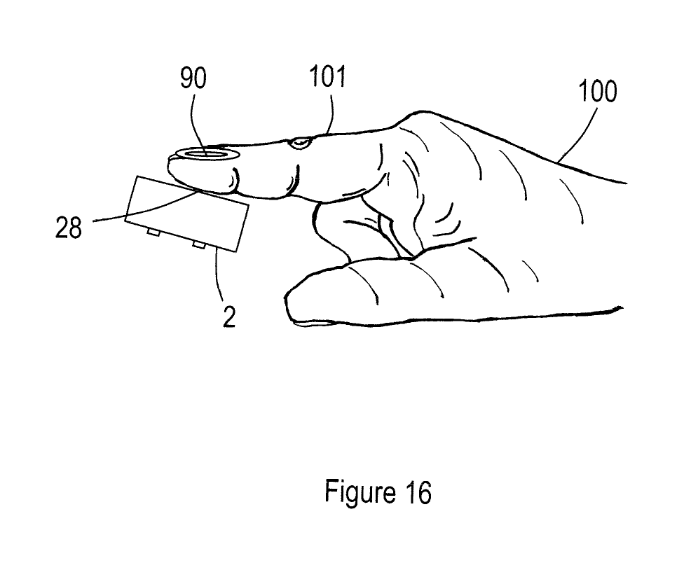

FIG. 11 shows a step by step operation of the AC/DC for MI apparatus 2 or 31. A user initially will undergo a complete body physical and medical assessment involving body dimensions along with body mass index calculations, heart rates, blood pressure measurements, cardiograms, and a body biochemical analysis including blood analyses, etc. to create a user health profile. The user's health profile is then loaded into the AC/DC for MI apparatus for use on a daily basis, particularly in regard to cardiac and heart information. Upon activation for daily use, the AC/DC for MI apparatus 2 or 31, performs monitoring tasks and if a user senses heart attack symptoms, he/she activates the Heart Attack Mode. The AC/DC for MI apparatus, either 2 or 31, then gives a series of prompts for response from the user. Firstly, a series of questions are posed: Do you have chest pains, etc. as detailed in FIG. 12. The User then responses by inputting either yes or no and the AC/DC for MI apparatus records the responses for processing and depending on the responses, the AC/DC for MI apparatus may then prompt the User to perform a cardiac ECG monitor test using various components disclosed and discussed in FIGS. 13-15. Similarly, the AC/DC apparatus may prompt the user to perform cardiac blood test as disclosed and discussed in FIGS. 16 to 19. Finally, the results of all of the prompts and/or tests may be electronically transmitted from AC/DC for MI apparatus 2 or 31 to appropriate medical contacts as previously discussed.

FIG. 12 shows the set of questions posed to a user a patient for a “Heart Attack Alert” prompted by the AC/DC for MI system, particularly “Do you have chest pains?”; “If yes, do they feel like a ‘brick’ is on your chest?”; If yes, do the pains radiate to your arms, particularly to your left arm?”; “If yes, do the pains radiate to your jaw?”; “Do you feel nauseas?”; Do you feel like vomiting?”; Do you feel dizzy?”; “Did you lose consciousness?”; “Are you short of breath?”. As previously disclosed in the discussion of FIG. 11, the responses to the above questions are recorded and processed by the AC/DC for MI apparatus to determine the appropriate follow-up prompt, if any.

FIG. 13 details components used in the event the answers to the prompts of FIG. 12 warrant further action regarding the AC/DC for MI system procedure. In essence, FIGS. 13, 13A, and 13B shows electrocardiographic (ECG) cardiac sensors and locations used to test for a heart attack with the AC/DC for MI system. FIG. 13B shows a top-view of an ECG cardiac sensor 90. ECG cardiac sensor 90 have a dual capability; it is capable of sensing electrocardiographic activity, in an electrocardiographic mode, and of sensing cardiac markers or evidence, in a cardiac marker or evidence mode; the use of the ECG cardiac sensor 90 in the cardiac sensing marker mode is discussed in reference to FIGS. 16 to 19 below. The AC/DC for MI system is provided with a number of EGC cardiac sensors, usually ten, which are configured to be placed at various locations on a user's body when used in an electrocardiographic mode; when used in the cardiac marker or evidence mode, only one ECG cardiac sensor is needed as is discussed in reference to FIGS. 16 to 19. FIG. 13C is a rear view of ECG cardiac sensor 90 which is provided with human skin adhesive on its toroid 92 enabling ECG cardiac sensor 90 to be attachable to a user. ECG cardiac sensor 90 also includes a microchip 94, also show in FIG. 13C, capable of sending and receiving signals to measure electrical activity of the heart and various arteries of a user. As seen in FIG. 13A, the various ECG cardiac sensors 90 are strategically placed on a user, several on the shoulder areas, a string across the chest area above the heart and several in a user's groin area.

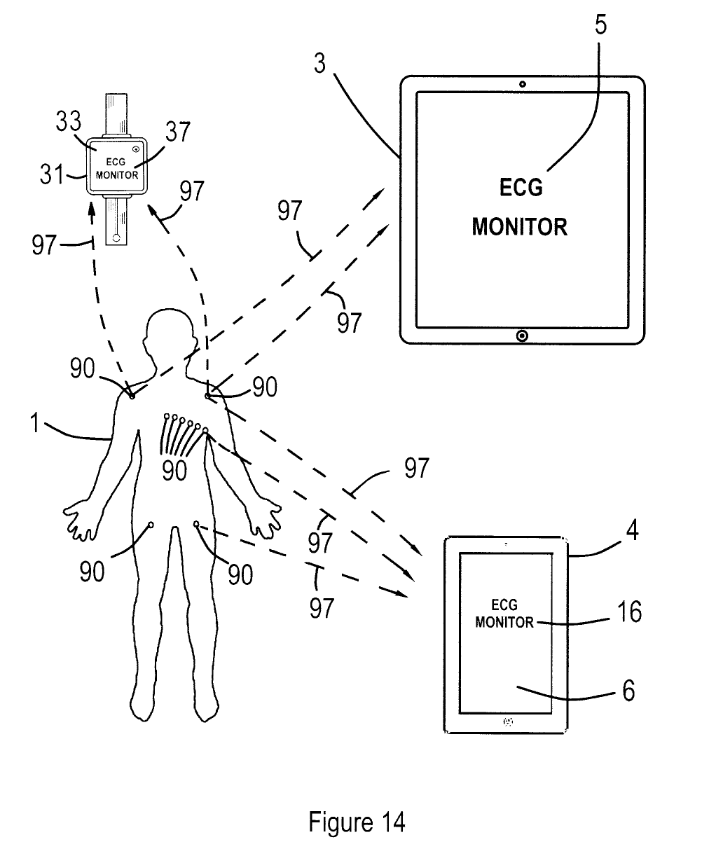

As shown in FIG. 14, the ECG cardiac sensors 90 are capable of sending and receiving signals from either AC/DC for MI apparatus 2 or 31 with receiving signals being processed by AC/DC for MI apparatus 2 or 31 apps to determine ECG activity in a user 1. Particularly shown in FIG. 14, are signals 97 being sent from sensors 90 to either electronic tablet 3 with “ECG MONITOR” display and/or to smartphone 5 with “ECG MONITOR” display. AC/DC for MI apparatus 31, with display screen 33 showing “ECG MONITOR” display, is also capable of receiving signals 97 from ECG cardiac sensors 90 and processing them to determine ECG activity of a user.

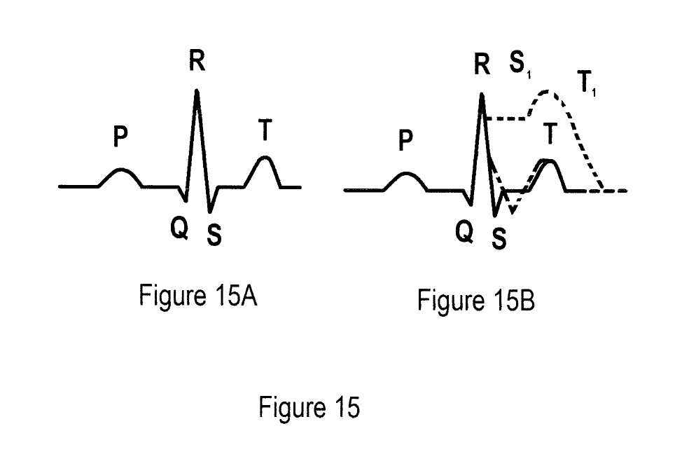

As shown in FIG. 15, the ECG activity reflected in the signals 97 are compared to the medical history ECG activity of a user, as shown in FIG. 15, to assess the information regarding the potential heart attack. FIG. 15A reflects an ECG pattern from the User's medical history, usually a base reference pattern, and is compared to the pattern shown in FIG. 15B reflecting the test current pattern. The apps of either AC/DC for MI apparatus 2 or 31 can process the ECG patterns to make an assessment as to heart attack activity.

FIG. 16 shows the final manner of how the AC/DC for MI system can be used for determining if a user is having, or has recently had a cardiac infarction. In this manner the AC/DC for MI apparatus 2 sensor probe 28 is used as a blood sensor and analyzer to sense for cardiac markers or evidence symptomatic of a heart attack i.e., proteins such as troponins, myoglobins, etc. released into a user's bloodstream as a result of myocardial ruptures. A ECG cardiac sensor 90, as previously described in FIG. 13, is capable of receiving and sending signals through a finger 101 of a hand of a user 100, the signals being generated by sensor 28 of AC/DC for MI apparatus 2; these signals are capable of sensing the presence of various cardiac markers or evidence, i.e., troponin, myoglobin, creatine kinase, e.g., in a user's blood stream. The presence, or absence, of any or all of these markers are sensed by sensors 90 and 28, processed by app 65 capable of analyzing the cardiac marker or evidence signals, of AC/DC for MI apparatus 2 microchip 60; the result of the cardiac marker or evidence analysis is then sent to either AC/DC electronic tablet 3 and/or 4 for processing as an assessment of the presence or absence of a heart attack of a user.

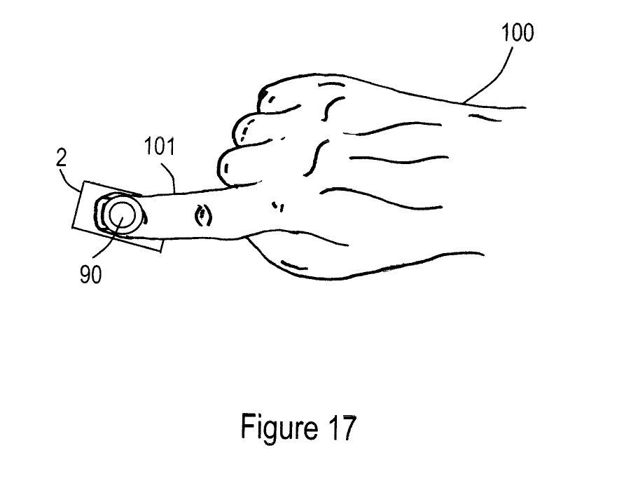

FIG. 17 shows a top view of the ECG cardiac sensor 90 and AC/DC apparatus 2 used to sense cardiac markers or evidence, of a finger 101 of on the hand 100 of a user, for a heart attack. with the AC/DC for MI system.

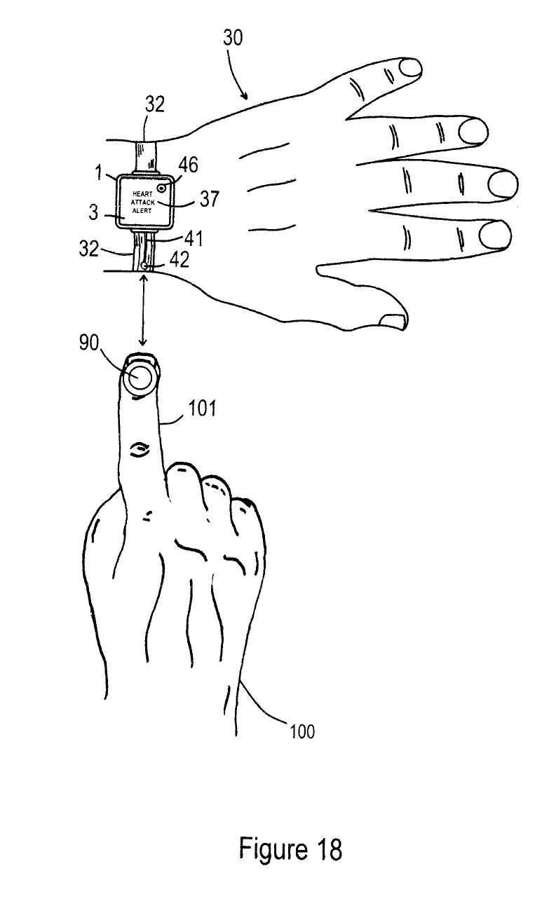

FIG. 18 shows a top view of the AC/DC for MI apparatus 31 for sensing cardiac markers or evidence in a user wherein ECG cardiac sensor 90 is placed on the top of finger 101 of a user. Subsequently, finger 101 is then placed on top of AC/DC for MI apparatus 31 sensor 42, capable of sending and receiving signals for analyzing cardiac markers or evidence, i.e., troponin, myoglobin, creatine kinase, etc. in a user's blood stream along with ECG cardiac sensor 90 used with AC/DC for MI apparatus 31. AC/DC for 31 is provided with a similar app as AC/DC apparatus 2 for processing ECG cardiac sensor signals to determine the presence of cardiac markers. The presence, or absence, of any or all of these markers are sensed by ECG cardiac sensors 90 and AC/DC for MI sensor 42 which are then processed by AC/DC for MI apparatus 31 as an assessment of the presence or absence of a heart attack of the User.

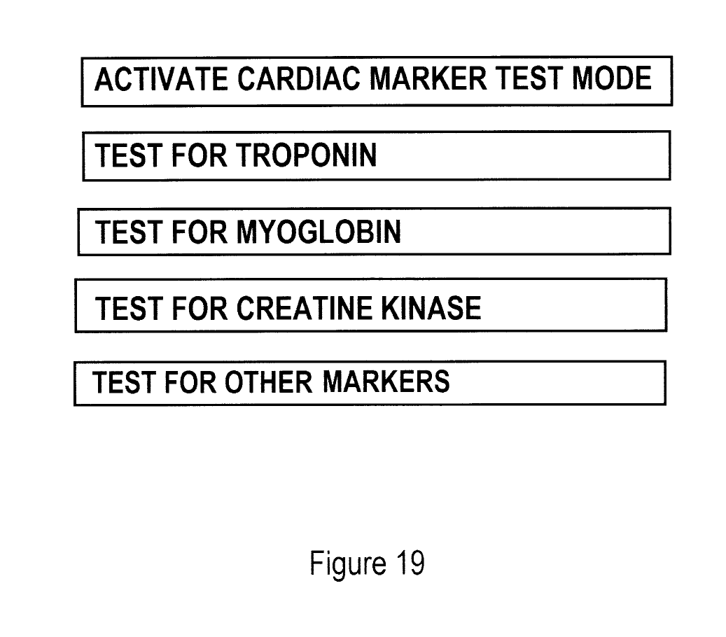

FIG. 19 shows the various blood biochemicals tested for a heart attack using the respective ECG sensors used to test for a heart attack with the AC/DC for MI system. Particularly, FIG. 19 shows the various cardiac markers or evidence which AC/DC for MI apparatuses 2 and 31 are capable of sensing, i.e., troponin, myoglobin, and creatine kinase.

In the event, two of the three modes described above indicate that a user is having, or recently has had a myocardial infarction, the respective AC/DC for MI apparatus 2 or 31 will alert medical staff or other authorities as to the condition of the user. In this manner the AC/DC for MI system can aid in furthering the care of a user.

When introducing elements of the present invention or the preferred embodiment(s) thereof, the articles “a”, “an”, “the”, and “said” are intended to mean there are one or more of the elements. The terms “comprising”, “including”, and “having” are intended to be inclusive and mean that there may be additional elements other than the listed elements. Additionally, any description of the exemplary or preferred embodiments is intended to be read in connection with the accompanying drawings, which are to be considered part of the entire written description.

Additionally, the principles of the invention could be practiced by those of skilled in the art with equivalent alternative constructions. Although the present invention has been described in considerable detail with reference to a certain preferred embodiment thereof, other embodiments are possible. Therefore, the spirit and scope of the appended claims should not be limited to the description of the preferred embodiment(s) contained herein. The invention may be embodied and practiced in other specific forms without departing from the spirit and essential characteristics thereof. The present embodiments are therefore to be considered in all respects as illustrative and not restrictive, the scope of the invention being indicated by the appended claims rather than by the foregoing description; and all variations, substitutions and changes which come within the meaning and range of equivalency of the claims are therefore intended to be embraced therein.

What is claimed:

1: A assisted capacity device calculator for myocardial infarction system for assisting a user during a myocardial infarction comprising
an apparatus provided with means for wirelessly sending and receiving an electronic signal and wherein
the apparatus is capable of being activated by the user and wherein
the apparatus is provided with a computer processing application software which can process any electronic signal received by the apparatus and wherein
the system includes a sensor component which is capable of sensing electro cardio activity or cardiac marker activity in the blood of the user and wherein
the system includes a sensor of sensing electro cardio activity or a cardio marker activity in the blood of the user and wherein
the sensor co-operates with the sensor component and wherein
the computer processing application software is capable of producing a visual display to inform or command the user to respond to a question for assessing whether the user is having or recently had a myocardial infarction.

2: The assisted capacity device calculator for myocardial infarction system of claim 1 wherein the apparatus is further provided with means for transmitting to and receiving a wireless signal from a global navigational positioning system.

3: The assisted capacity device calculator for myocardial infarction system of claim 2 wherein data sensed by the sensor component is capable of being wirelessly transmitted to the apparatus.

4: The assisted capacity device calculator for myocardial infarction system of claim 3 wherein the system is provided with a mobile electronic appliance and wherein
the computer processing application software is capable of producing a question regarding the user's cardiac condition wherein the question can be displayed on the mobile electronic appliance and wherein the computing processing application software is capable of analyzing a response to the question.

5: The assisted capacity device calculator for myocardial infarction system of claim 4 wherein the apparatus is capable of having the user input a response to the question using the mobile electronic appliance.

6: The assisted capacity device calculator for myocardial infarction system of claim 5 wherein the sensor component is capable of being attached to the body of the user.

7: The assisted capacity device calculator for myocardial infarction system of claim 6 wherein the computer processing application software is capable of analyzing data produced by the sensor component or sensor.

8: The assisted capacity device calculator for myocardial infarction system of claim 7 further wherein the data produced comprises electro cardio activity of the user.

9: The assisted capacity device calculator for myocardial infarction system of claim 8 further wherein the data produces an electrocardiogram.

10: The assisted capacity device calculator for myocardial infarction system of claim 9 wherein the computer processing application software is capable of analyzing the electrocardiogram relative to a reference electrocardiogram of the user.

11: The assisted capacity device calculator for myocardial infarction system of claim 7 further wherein the data produced comprises cardio marker activity of the user.

12: The assisted capacity device calculator for myocardial infarction system of claim 11 further wherein the cardio marker activity of the user comprises evidence of a cardiac protein.

13: The assisted capacity device calculator for myocardial infarction system of claim 12 further provided with a lanyard or necklace to allow the user to wear the apparatus.

14: The assisted capacity device calculator for myocardial infarction system of claim 13 wherein the apparatus is housed in a body having a substantially rectangular cuboid structure and wherein
the mobile electronic appliance is capable of receiving and sending electronic signals from and to the apparatus.

15: The assisted capacity device calculator for myocardial infarction system of claim 14 wherein the mobile electronic appliance comprises an electronic tablet.

16: The assisted capacity device calculator for myocardial infarction system of claim 14 wherein the mobile electronic appliance comprises a smartphone.

17: The assisted capacity device calculator for myocardial infarction system of claim 3 wherein the apparatus is housed in a watch.

18: A assisted capacity device calculator for myocardial infarction system for assisting a user during a myocardial infarction comprising
an apparatus provided with means for wirelessly sending and receiving an electronic signal and wherein
the apparatus is capable of being activated by the user and wherein
the apparatus is provided with a computer processing application software which can process any electronic signal received by the apparatus and wherein
the system includes a sensor component which is capable of sensing electro cardio activity or cardiac marker activity in the blood of the user and wherein
the system includes a sensor of sensing electro cardio activity or a cardio marker activity in the blood of the user and wherein the sensor co-operates with the sensor component and wherein
the computer processing application software is capable of producing a visual display to inform or command the user to respond to a question for assessing whether the user is having a myocardial infarction and wherein
the apparatus is capable of having the user input a response to the question and wherein
the apparatus is further provided with means for transmitting to and receiving a wireless signal from a global navigational positioning system and wherein
data sensed by the sensor component is capable of being wirelessly transmitted to the apparatus and wherein
the system is provided with a mobile electronic appliance having a screen display
and wherein the mobile electronic appliance is capable of receiving and sending electronic signals to the apparatus wherein
the computer processing application software is capable of producing a question regarding the user's cardiac condition wherein the question can be displayed on the screen display of the mobile electronic appliance and wherein
the computing processing application software is capable of analyzing a response to the question and wherein
the apparatus is capable of having the user input a response to the question wherein
the sensor component is capable of being attached to the body of the user and
wherein the computer processing application software is capable of analyzing data produced by the sensor component or sensor and wherein
the data produced comprises electro cardio activity of the user and wherein
the electro cardio activity data produces an electrocardiogram and wherein
the computer processing application software is capable of analyzing the electrocardiogram relative to a reference electrocardiogram of the user and wherein
the data produced comprises cardio marker activity of the user and wherein
the cardio marker activity of the user comprises evidence of a cardiac protein and wherein
the system is provided with a lanyard or necklace to allow the user to wear the apparatus and wherein
the apparatus is housed in a body having a substantially rectangular cuboid structure and wherein
the mobile electronic appliance and wherein the mobile electronic appliance comprises an electronic tablet or a smartphone wherein the user can use the system to determine if the user is having or recently had a cardiac infarction.

19: A assisted capacity device calculator for myocardial infarction system for assisting a user during a myocardial infarction comprising
an apparatus provided with means for wirelessly sending and receiving an electronic signal and wherein
the apparatus is capable of being activated by the user and wherein
the apparatus is provided with a computer processing application software which can process any electronic signal received by the apparatus and wherein
the system includes a sensor component which is capable of sensing electro cardio activity or cardiac marker activity in the blood of the user and wherein
the system includes a sensor of sensing electro cardio activity or a cardio marker activity in the blood of the user and wherein
the sensor co-operates with the sensor component and wherein
the apparatus is further provided with means for transmitting to and receiving a wireless signal from a global navigational positioning system and wherein
the data sensed by the sensor component is capable of being wirelessly transmitted to the apparatus and wherein
the system is housed in a watch capable of being worn by the user and wherein
the watch has a screen display and wherein
the computer processing application software is capable of producing a question regarding the user's cardiac condition wherein the question can be displayed on the screen display of the watch and wherein
the watch is capable of having the user input a response to the question and wherein
the computing processing application software is capable of analyzing the response to the question and wherein
the sensor component is capable of being attached to the body of the user and
wherein the computer processing application software is capable of analyzing data produced by the sensor component or sensor and wherein
the data comprises electro cardio activity data of the user and wherein
the electro cardio activity data produces an electrocardiogram wherein
the computer processing application software is capable of analyzing the electrocardiogram relative to a reference electrocardiogram of the user and wherein
the data produced comprises cardio marker activity of the user and wherein
the cardio marker activity of the user comprises evidence of a cardiac protein and wherein the user can use the system to determine if the user is having or recently had a cardiac infarction.
